(19) United States
(12) Patent Application Publication
(10) Pub. No.: US 2024/0382161 A1
(43) Pub. Date: Nov. 21, 2024

(54) Method and System for Post-Partum Haemorrhage Detection

(71) Applicant: Baymatob Pty Ltd, Sydney (AU)

(72) Inventors: Sarah Catherine McDonald, Sydney (AU); Rishi Ramakrishnan, Sydney (AU)

(73) Assignee: Baymatob Pty Ltd, Sydney (AU)

(21) Appl. No.: 18/688,620

(22) PCT Filed: Sep. 2, 2022
(86) PCT No.: PCT/AU2022/051075

§ 371 Date: Mar. 1, 2024

(30) Foreign Application Priority Data
Sep. 2, 2021 (AU) 2021902850

(51) Int. Cl. A61B 5/00; A61B 5/01; A61B 5/145; A61B 5/256; A61B 5/257; A61B 5/282; A61B 5/296; A61B 90/00

(52) U.S. Cl. CPC A61B 5/7267; A61B 5/01; A61B 5/14517; A61B 5/256; A61B 5/257; A61B 5/282; A61B 5/296; A61B 5/4343; A61B 5/6823; A61B 5/6832; A61B 5/725; A61B 5/7275; A61B 5/742; A61B 90/06; A61B 2090/064; A61B 2560/0468; A61B 2562/0219

(57) ABSTRACT

Disclosed is a monitoring system configured for determining risk of postpartum haemorrhage to a patient. The monitoring system comprises an electrical potential sensor for collecting patient data and at least one electrode for attaching the electrical potential sensor to a body of the patient. The monitoring system also comprises a communications module for transmitting the patient data to a detection controller, wherein the detection controller is configured to determine the risk of the postpartum haemorrhage based on the patient data.

### TECHNICAL FIELD

The present disclosure relates to detecting postpartum haemorrhage.

### BACKGROUND

Within the United States, postpartum haemorrhage (PPH) is one of the leading causes of maternal deaths. responsible for around 11% of deaths. Globally. postpartum haemorrhage is the leading cause of maternal deaths accounting for approximately a quarter of all deaths. Many maternal deaths due to obstetric haemorrhage are believed to be preventable. Unfortunately, mortality rates associated with postpartum haemorrhage have shown no overall improvement over the last forty years and the prevalence of the condition is increasing.

Currently, there is no method to identify. in advance, which women in labour will develop postpartum haemorrhage and diagnosis only happens after postpartum haemorrhage has occurred. Clinically, postpartum haemorrhage is diagnosed through observation and estimation of the quantity of blood loss. The imprecise estimation of blood loss is a leading cause of delayed response to postpartum haemorrhage. Most deaths from postpartum haemorrhage are classified as avoidable as the use of prophylactic uterotonics during the third stage of labour, as well as timely and appropriate management, would save many lives.

The impacts of postpartum haemorrhage extend beyond mortality. Women who suffer postpartum haemorrhage and survive are at risk of suffering irreversibly debilitating conditions including multi-organ failure, complications of multiple blood transfusions, peripartum hysterectomy and unintended damage to pelvic organs, loss of fertility and psychological sequelae, including posttraumatic stress disorders. The prevalence of these morbidities and associated interventions have been increasing. Postpartum haemorrhage events requiring blood transfusions increased from around 8 per 10,000 deliveries in 1993 to around 40 per 10,000 deliveries in 2014 in the United States. Postpartum hemorrhage events requiring a procedure other than blood transfusion have risen from 0.9 per 1,000 in 2001-2002 to 1.9 per 1,000 in 2011-2012 in the United States.

It is desired to address or ameliorate one or more disadvantages or limitations associated with the prior art, or to at least provide a useful alternative.

### SUMMARY

This Summary is provided to introduce a selection of concepts in a simplified form that are further described below in the Detailed Description. This Summary is not intended to identify essential features of the claimed subject matter, nor is it intended to be used to limit the scope of the claimed subject matter.

Disclosed is a monitoring system configured for determining risk of postpartum haemorrhage to a patient, the monitoring system comprising: an electrical potential sensor for collecting patient data; at least one electrode for attaching the electrical potential sensor to a body of the patient; and a communications module for transmitting the patient data to a detection controller, wherein the detection controller is configured to determine the risk of the postpartum haemorrhage based on the patient data.

The electrical potential sensor may include a sensor selected from the set of sensors consisting of an electromyography (EMG) sensor, an electrohepatogram (EHG) sensor and electrocardiogram (ECG) sensor.

The monitoring system may further comprise a movement sensor.

The movement sensor may include a sensor selected from the set of sensors consisting of an accelerometer and a gyroscope.

The monitoring system may further comprise a deformation sensor.

The deformation sensor may include a sensor selected from the set of sensors consisting of a flex sensor and a stretch sensor.

The monitoring system may further comprise a patient response sensor.

The patient response sensor may include a sensor selected from the set of sensors consisting of a temperature sensor and a sweat sensor.

The detection controller may be configured to apply a machine learning model to the patient data to determine the risk of postpartum haemorrhage.

The machine learning model may be a nonlinear model.

The detection controller may be configured to apply the machine learning model to the patient data to determine the risk of the postpartum haemorrhage by estimating a likelihood of the postpartum haemorrhage.

The estimated likelihood may be compared to a predetermined threshold to determine if postpartum haemorrhage is likely.

The detection controller may include a trained classifier configured to determine the risk of the postpartum haemorrhage.

The electrical potential sensor, the at least one electrode, the communications module and the detection controller may be located within a housing.

The detection controller may be located separately from the electrical potential sensor.

The monitoring system may have a positive predictive value at least 70%

The monitoring system may have a negative predictive value of at least 70%.

The detection controller may be configured to automatically perform a method comprising: receiving the patient data from the monitoring device; estimating a likelihood of the postpartum haemorrhage by processing the patient data from to form a plurality of descriptors for data points in the patient data, the plurality of descriptors being processed by a machine learning model to estimate the likelihood; determining a postpartum haemorrhage risk for the patient by comparing the estimated likelihood to a predetermined threshold; and displaying the determined postpartum haemorrhage risk to an operator.

Disclosed is a method of detecting a high risk of postpartum haemorrhage in a patient, the method comprising: collecting patient data from a monitor having a plurality of medical electrode members attached to a body of a patient, the patient data being collected from at least one sensor type; estimating a likelihood of the postpartum haemorrhage by processing the patient data from the at least one sensor type to form a plurality of descriptors for data points in the patient data, the plurality of descriptors being processed by a machine learning model to estimate the likelihood; and displaying the determined postpartum haemorrhage risk to an operator.

The method may further comprise: determining a postpartum haemorrhage risk for the patient by comparing the estimated likelihood to a predetermined threshold;

### BRIEF DESCRIPTION OF FIGURES

At least one embodiment of the present invention is described, by way of example only, with reference to the accompanying figures.

FIG. 1 illustrates a functional block diagram of an example processing system that can be utilised to embody or give effect to a particular embodiment;

FIG. 2 illustrates an example network infrastructure that can be utilised to embody or give effect to a particular embodiment;

FIGS. 3A and 3B illustrate a flowchart of the use of an electrode assembly to detect postpartum haemorrhage;

FIG. 4 illustrates an example of an electrode assembly;

FIG. 5 illustrates an example of a medical device that can be used together with the electrode assembly of FIG. 4;

FIGS. 6A and 6B illustrate an exemplary method of using the electrode assembly;

FIG. 7 illustrates a device station for the medical device of FIG. 5;

FIG. 8 illustrates processing patient data for a postpartum haemorrhage detection method;

FIG. 9 illustrates a postpartum haemorrhage detection method;

FIG. 10 illustrates an alternative postpartum haemorrhage detection method;

FIG. 11 illustrates an alternative postpartum haemorrhage detection method; and

FIG. 12 illustrates a PPH monitoring system according to one embodiment.

### DETAILED DESCRIPTION

The following modes, given by way of example only, are described in order to provide a more precise understanding of one or more embodiments. In the figures, like reference numerals are used to identify like parts throughout the figures.

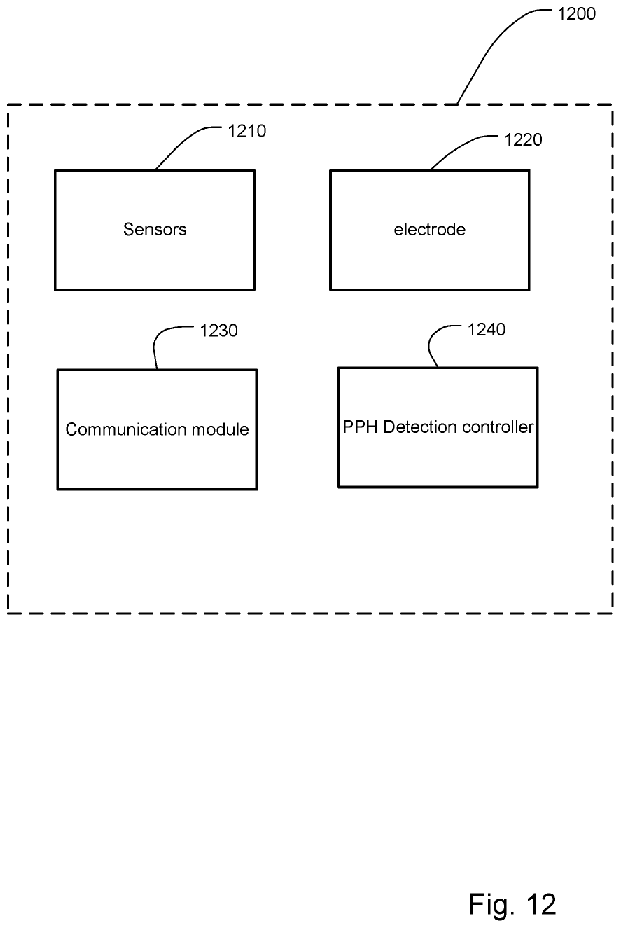

FIG. 12 shows a PPH monitoring system 1200 according to at least one embodiment of the present disclosure that may be used to determine a risk of postpartum haemorrhage to a patient. The PPH monitoring system 1200 has one or more sensors 1210. The sensors may be one or more of an electrical potential sensor, a movement sensor, a deformation sensor and/or a patient response sensor. Some of the sensors 1210, such as an electrical potential sensor, may be attached to one or more electrodes 1220 for attaching the one or more sensors 1210 to a body of the patient.

The one or more sensors 1210 collect patient data that may be transmitted by a communication module 1230 to a PPH detection controller 1240. In one example, components of the PPH monitoring system 1200, including the sensors 1210, electrodes 1220, communication module 1230 and the communication module 1230, may be located in a housing (thus forming a monitoring device), e.g., a water proof and impact resistant housing for use during labour. Alternatively, some components of the PPH monitoring system 1200 may be located separately to the housing. In one example the PPH detection controller 1240 may be located separately from the housing.

The communication module 1230 may control communications within the housing and send the patient data collected from the sensors 1210 to the PPH detection controller 1240 via a communications bus. Alternatively, when the PPH detection controller 1240 is located separately from the housing, the communication module 1230 may communicate with the PPH detection controller 1240 using wireless or wired communications. The PPH detection controller 1240 may be executed on and/or embodied in a standalone computing device such as a laptop, tablet or smartphone.

The PPH detection controller 1240 is configured to apply a machine learning model to the patient data to determine a risk of postpartum haemorrhage. The risk of postpartum haemorrhage may be determined by estimating a likelihood of the postpartum haemorrhage.

Disclosed is a monitoring system configured for determining risk of postpartum haemorrhage to a patient. The monitoring system includes an electrical potential sensor for collecting patient data. The monitoring system also includes at least one electrode for attaching the electrical potential sensor to a body of the patient as well as a communications module for transmitting the patient data to a detection controller. The detection controller is configured to determine the risk of the postpartum haemorrhage based on the patient data. A postpartum haemorrhage risk for the patient is determined by comparing the estimated likelihood to a predetermined threshold and then displayed to an operator. In one example. the monitoring system may be a monitoring device.

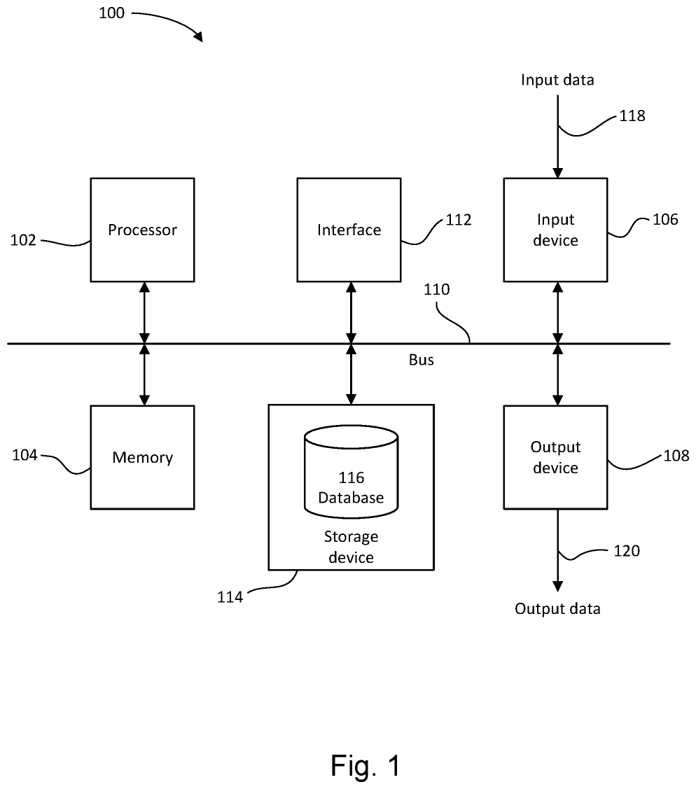

A particular embodiment of the PPH monitoring system, or at least one or more components thereof, can be realised using a processing system, an example of which is shown in FIG. 1. In particular, the processing system 100 generally includes at least one processor 102, or processing unit or plurality of processors, memory 104, at least one input device 106 and at least one output device 108, coupled together via a bus or group of buses 110. In certain embodiments, input device 106 and output device 108 could be the same device. An interface 112 can also be provided for coupling the processing system 100 to one or more peripheral devices, for example interface 112 could be a PCI card or PC card. At least one storage device 114 which houses at least one database 116 can also be provided. The memory 104 can be any form of memory device, for example, volatile or non-volatile memory, solid state storage devices, magnetic devices, etc. The processor 102 could include more than one distinct processing device, for example to handle different functions within the processing system 100.

Input device 106 receives input data 118 and can include, for example, a keyboard, a pointer device such as a pen-like device or a mouse, audio receiving device for voice controlled activation such as a microphone, data receiver or antenna such as a modem or wireless data adaptor, data acquisition card, etc. Input data 118 could come from different sources, for example keyboard instructions in conjunction with data received via a network. Output device 108 produces or generates output data 120 and can include, for example, a display device or monitor in which case output data 120 is visual, a printer in which case output data 120 is printed, a port for example a USB port, a peripheral component adaptor, a data transmitter or antenna such as a modem or wireless network adaptor, etc. Output data 120 could be distinct and derived from different output devices, for example a visual display on a monitor in conjunction with data transmitted to a network. A user could view data output, or an interpretation of the data output, on, for example, a monitor or using a printer. The storage device 114 can be any form of data or information storage means, for example, volatile or non-volatile memory, solid state storage devices, magnetic devices, etc.

In use, the processing system 100 is adapted to allow data or information to be stored in and/or retrieved from, via wired or wireless communication means, the at least one database 116. The interface 112 may allow wired and/or wireless communication between the processing unit 102 and peripheral components that may serve a specialised purpose. The processor 102 receives instructions as input data 118 via input device 106 and can display processed results or other output to a user by utilising output device 108. More than one input device 106 and/or output device 108 can be provided. It should be appreciated that the processing system 100 may be any form of terminal, server, specialised hardware, or the like.

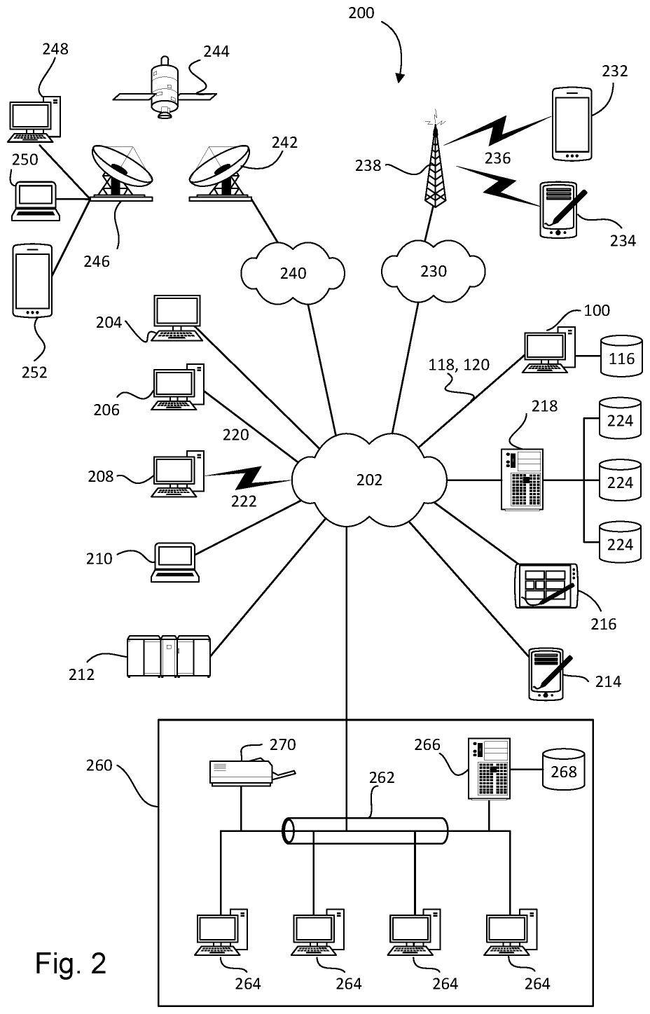

The processing system 100 may be a part of a networked communications system 200, as shown in FIG. 2. Processing system 100 could connect to network 202, for example the Internet or a WAN. Input data 118 and output data 120 could be communicated to other devices via network 202. Other terminals, for example, thin client 204, further processing systems 206 and 208, notebook computer 210, mainframe computer 212, PDA 214, pen-based computer or tablet 216, server 218, etc., can be connected to network 202. A large variety of other types of terminals or configurations could be utilised. The transfer of information and/or data over network 202 can be achieved using wired communications means 220 or wireless communications means 222. Server 218 can facilitate the transfer of data between network 202 and one or more databases 224. Server 218 and one or more databases 224 provide an example of an information source.

Other networks may communicate with network 202. For example, telecommunications network 230 could facilitate the transfer of data between network 202 and mobile, cellular telephone or smartphone 232 or a PDA-type device 234, by utilising wireless communication means 236 and receiving/transmitting station 238. Satellite communications network 240 could communicate with satellite signal receiver 242 which receives data signals from satellite 244 which in turn is in remote communication with satellite signal transmitter 246. Terminals, for example further processing system 248, notebook computer 250 or satellite telephone 252, can thereby communicate with network 202. A local network 260, which for example may be a private network, LAN, etc., may also be connected to network 202. For example, network 202 could be connected with Ethernet 262 which connects terminals 264, server 266 which controls the transfer of data to and/or from database 268, and printer 270. Various other types of networks could be utilised.

The processing system 100 is adapted to communicate with other terminals, for example further processing systems 206, 208, by sending and receiving data, 118, 120, to and from the network 202, thereby facilitating possible communication with other components of the networked communications system 200.

Thus, for example, the networks 202, 230, 240 may form part of, or be connected to, the Internet, in which case, the terminals 206, 212, 218, for example, may be web servers, Internet terminals or the like. The networks 202, 230, 240, 260 may be or form part of other communication networks, such as LAN, WAN, Ethernet, token ring, FDDI ring, star, etc., networks, or mobile telephone networks, such as GSM, CDMA, 4G, 5G etc., networks, and may be wholly or partially wired, including for example optical fibre, or wireless networks, depending on a particular implementation.

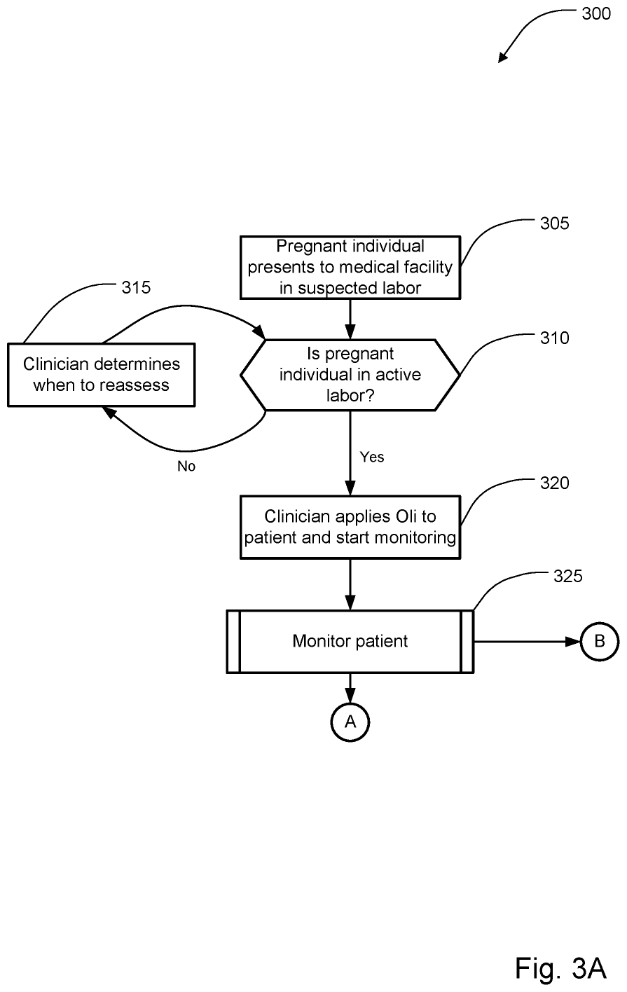

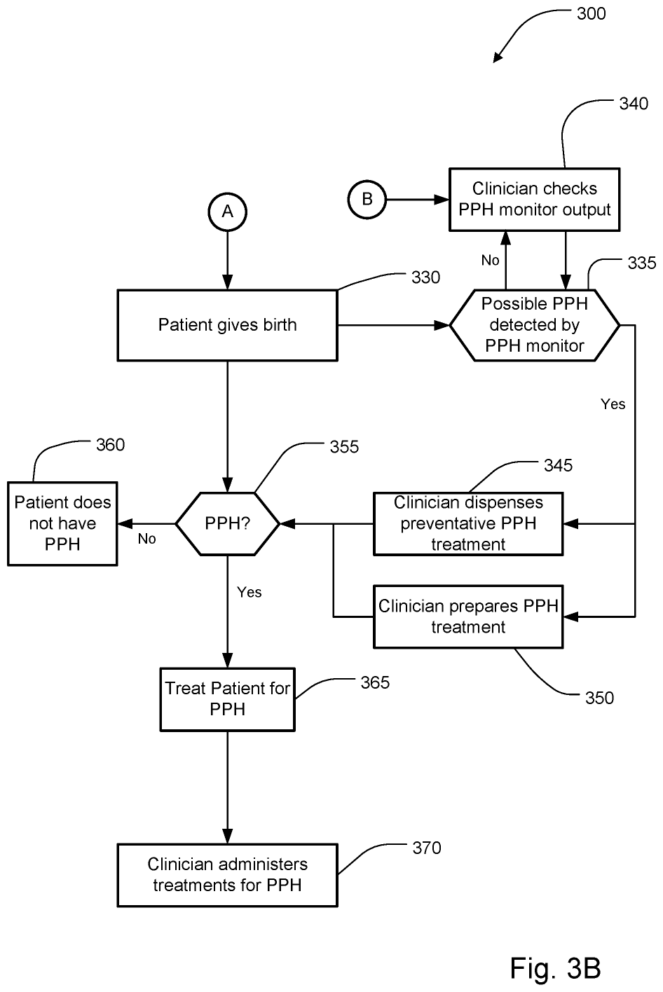

FIGS. 3A and 3B show a clinical environment workflow 300 in which a PPH detection method can be performed. The clinical environment workflow 300 starts with a patient presentation 305 where a pregnant individual presents to a medical facility and is suspected of being in labour. At labour check 310 a check is made to determine if the patient is in active labour. If the patient is not in active labour a clinician review 315 may be scheduled to reassess the state of the patient at a later time. If the patient is in labour, the workflow progresses to apply a PPH monitor 320 where a PPH monitor, such as the medical device to be described herein, is applied/attached to the patient, e.g., to the abdomen of the patient. The PPH monitor provides patient data that can be analysed by a PPH detection controller to determine if postpartum haemorrhage is likely. Next, PPH monitoring 325 of the patient commences. The PPH monitoring 325 will be described in more detail below where a PPH detection method is described. As the patient is monitored the workflow progresses as a number of events happen, possibly in parallel. As the patient is monitored, a clinician continues monitoring for PPH 340 by checking output from the PPH detection controller. The patient may also give birth 330 at which point a PPH occurred check 355 may take place by the clinician using traditional means. If PPH has not occurred then the patient does not have PPH 360 and no treatment is required. If PPH occurred, the clinical environment workflow 300 moves to treat the patient for PPH 365.

As the patient gives birth 330 the PPH monitor may continue to collect data from the patient at a possible PPH detected stage 335. If no PPH is detected by the possible PPH detected stage 335 then the patient continues to be monitored by the clinician at the continue monitoring for PPH stage 340 where the clinical environment workflow 300 may return to the possible PPH detected 335. If the PPH monitor detects PPH, then a clinician may take action, such as dispense preventative PPH treatment 345 and/or prepare for PPH treatment 350 to be carried out at the treat patient with PPH stage 365.

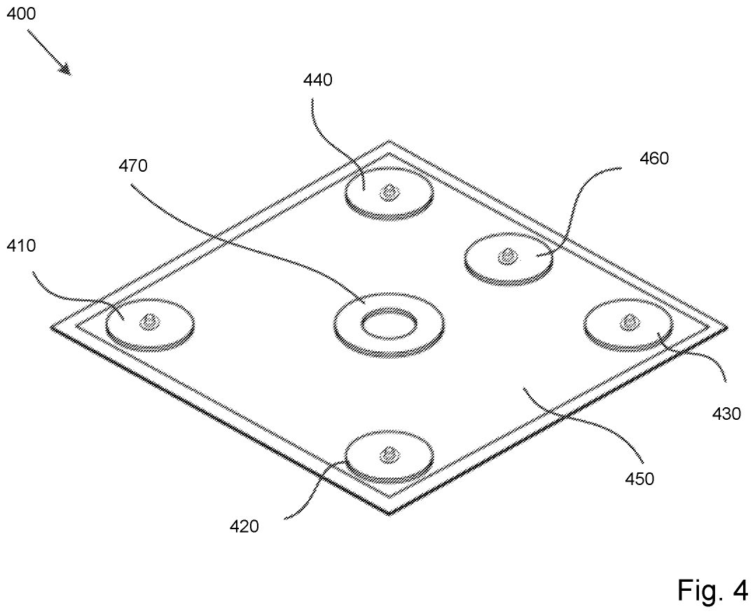

Referring to FIG. 4, there is provided an example of an electrode assembly 400 (which may also be referred to as an “electrode sheet”), which includes a plurality of medical electrode members 410, 420, 430 and 440; and at least one covering sheet 450 removably attached to the plurality of medical electrode members 410, 420, 430 440 and 460. Each of the medical electrode members 410, 420, 430, 440 and 460 may have the same structure as a known medical electrode device.

In this example, the medical electrode member 410 includes a flexible sheet (also referred to as an “electrode backing”) adaptable to the contour of the skin of a patient. The flexible sheet is made of an insulating material, e.g., cloth, plastic, closed cell foam, or any other suitable insulating material that does not conduct electrically, e.g., the electrode backing material can be a foam-and-plastic combination including an adhesive flexible seal that is adhered on top of the flexible sheet around an electrode connector.

A non-electrode adhesive pad 470 is located at or proximate to the centre of the covering sheet 450. Alternatively, the non-electrode adhesive pad 470 may be located in any other location on the covering sheet 450 that allows supporting the medical device. Further, the non-electrode adhesive pad 470 may have any other shape, as long as it allows the non-electrode adhesive pad 470 to adhere to the patient's body and support the medical device on attachment.

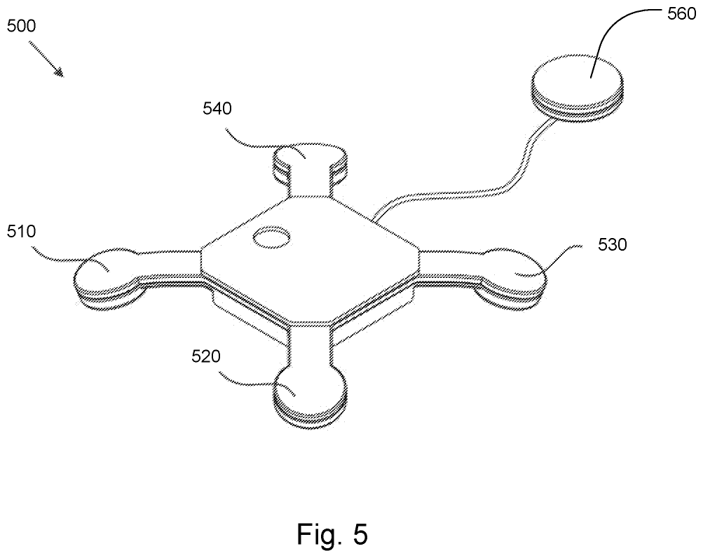

Referring now to FIG. 5 where an example is provided of a medical device 500 that can be used together with the electrode assembly 400 shown in FIG. 4, an example of which is the OLI (TM) device to which a plurality of electrode members 410 can be fastened and is described in Australian Provisional Patent Application No. 2016905046 titled “Apparatus for monitoring pregnancy or labour” and/or in PCT Application No. PCT/AU2017/051346 of the same name. The assembly of the medical device 500 and the electrode assembly 400 creates a PPH monitor that attaches to a patient. The medical device 500 typically has a power source, such as a battery, to power internal components. The power source may also be used to power a communications system in the medical device 500 that may communicate wirelessly or over wires with a PPH detection controller.

The medical device 500 includes a plurality of electrode connecting portions 510. 520, 530, 540 and 560. Each of the electrode connecting portions is adapted to be connected to a corresponding one of the medical electrode members 410, 420, 430, 440 and 460. Accordingly, the relative positions of the medical electrode members 410, 420, 430, 440 and 460 in the electrode assembly 400 may be arranged based on the relative positions of the corresponding electrode connecting portions 510, 520, 530, 540 and 560 on the medical device 500. As such, it would be understood by the skilled addressee that variations to the shape and arrangement of the features of the of the corresponding electrode connecting portions 510, 520, 530, 540 and 560 in accounting for relative positions of the medical electrode members 410, 420, 430, 440 is within the scope of the invention as described and defined in the claims.

The electrode members 410, 420, 430, 440 and 460 are mutually spaced apart in the electrode assembly 400 to mitigate mechanical and electrical interference between adjacent ones of the electrode members 410, 420, 430, 440. The electrode members 410, 420, 430, 440 are also spaced apart to connect to selected points on the skin, depending on the particular medical procedure and medical device 500. Example dimensions of the electrode assembly 400 can be about 100 millimetres (mm) between electrode members along each side, i.e., a square with sides over 100 mm. Example spacings of the electrode members 410, 420, 430, 440 can be over 50 mm centre-to-centre, e.g., 100 mm between centres, e.g., about 100 mm between centres along each side of the square arrangement in FIG. 4.

The term “patient” used in this disclosure includes both human and animal patients and users. Accordingly, the medical device 500 may include medical devices, well-being equipment and sport-monitoring equipment, for humans or veterinary devices for animals.

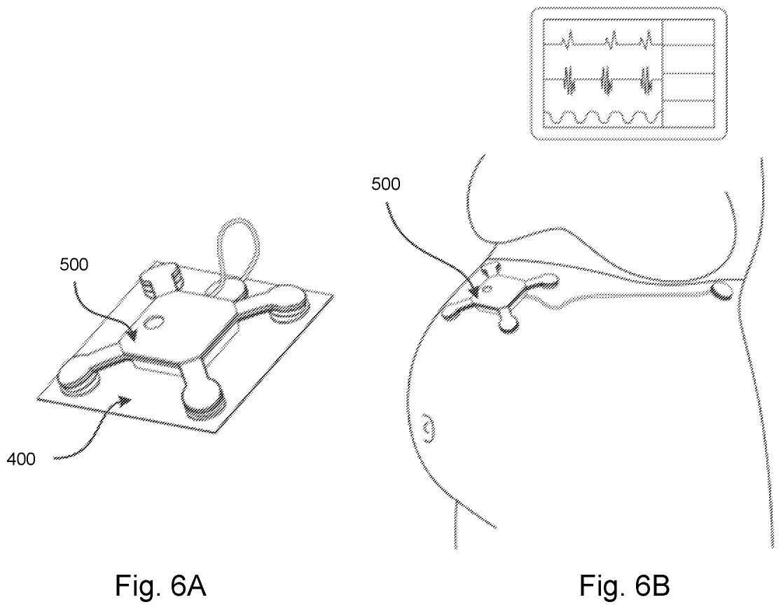

A user can attach the electrode assembly 400 to the medical device 500, as shown in FIG. 6A, to form a PPH monitor. This may occur by pressing the electrode assembly 400 and the medical device 500 together. The electrode assembly 400 may be connected to the medical device 500 so that each of the medical electrode members are secured to a corresponding one of the electrode connecting portions. The user may fasten the fastener (made of a conductive material (e.g., metal), and of a fastener type including a snap fastener (male or female), a tab, a wire or a custom connector) of an electrode connector of each medical electrode member (410, 420, 430, 440 or 460) to a cooperating fastener (which is made of a conductive material (e.g., metal), and of a cooperating type to the fastener's type, e.g., a snap fastener (female or male), a tab, a wire or a custom connector respectively) on a corresponding electrode connecting portion of the medical device 500.

Alternatively, or additionally, the user may adhere the flexible sheet of each medical electrode member (410, 420, 430, 440, or 460) to a flat surface on the corresponding electrode connecting portion of the medical device 500. In addition, the user may further fasten or adhere the non-electrode adhesive pad 470 to a corresponding electrode connecting portion of the medical device 500. In a further embodiment, the method may further include the step of detaching a perforated section of the second covering sheet that is connected to at least one of the plurality of medical electrode members and attaching the at least one of the electrode assembly to a patient's body or a medical device.

The user peels off or removes the second covering sheet from the electrode assembly 100, to expose a patient-side adhesive layer of each medical electrode member (410, 420, 430, 440 or 460), and the patient-side adhesive layer of the non-electrode adhesive pad 470. The user then attaches the medical device 500 with the plurality of medical electrode members to a patient's body such that the plurality of medical electrode members are secured to the patient's body. For example, the medical device 500 with the medical electrode members 410, 420, 430, 440, 460 and the patient-side adhesive layer of the non-electrode adhesive pad 470 is applied to the patient's body as shown in FIG. 6B, e.g., to the abdomen. The medical device 500, along with the medical electrode members 410, 420, 430, 440, 460 and the non-electrode adhesive pad 470 are secured to the patient's body through the adhesive layer of each medical electrode member and the adhesive layer of the non-electrode adhesive pad 470.

After being secured to the patient's body, the medical device 500 and the medical electrode members 410, 420, 430, 440 and 460 can be used to monitor or stimulate the patient, as shown in FIG. 6B. The medical device 500 monitors the electrical signals captured by the medical electrode members 410, 420, 430, 440 and 460, or outputs electrical signals to the medical electrode members 410, 420, 430, 440 and 460 for stimulating the patient's body. The PPH monitor may collect patient data using as an electrical potential sensor, such as an EMG sensors and/or other sensors, such as a patient response sensor implemented as a temperature sensor operating through the electrode member 410, 420, 430, 440 and 460. Other sensors may be built in to the medical device 500 such as a movement sensor implemented as an accelerometer. Additional patient response sensors may be built into the PPH monitor. After use, the medical device 500 and the medical electrode members 410, 420, 430, 440 or 460 can be removed from the patient's body

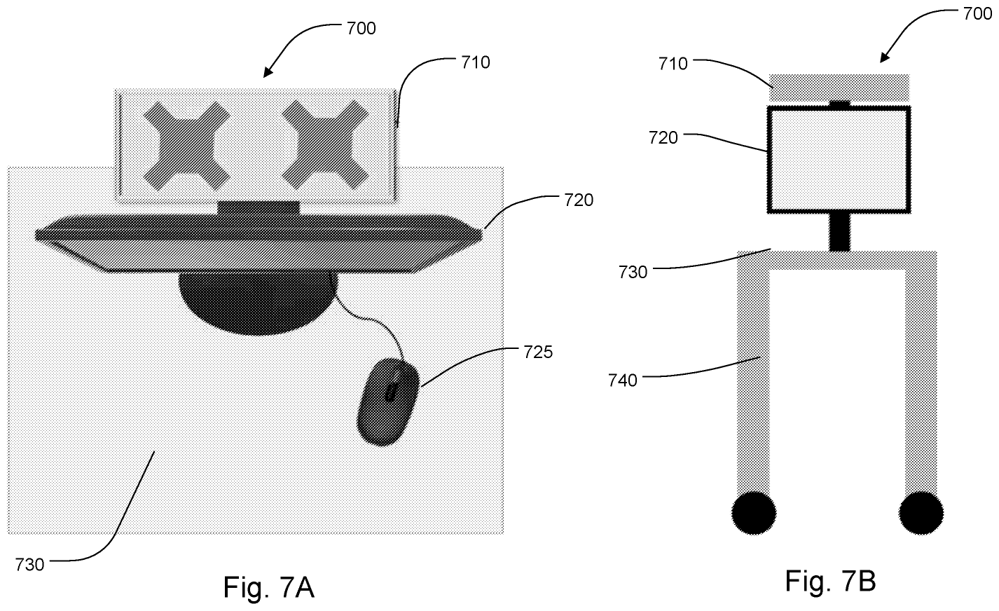

A device station 700 will now be described with reference to FIGS. 7A and 7B. The device station 700 provides a charging location and storage for a PPH monitor comprising a medical device, such as medical device 500, and an electrode assemble such as the electrode assembly 400. The device station 700 also has a user interface for a user of the postpartum haemorrhage detection system and a preparation area for assembling the PPH monitor.

The device station 700 has a charge station 710 where an integrated power storage module, e.g., a battery pack, for a medical device component of a PPH monitor may be charged. The charge station 710 may also include data transfer capabilities to allow one or more medical devices to be configured for wireless communication with the device station 700. In one example, a medical device and the device station 700 may be paired for Bluetooth communication when the medical device is connected to the charge station 710. Alternatively, the medical device may be configured to communicate using other wireless communication protocols such as a Wi-Fi protocol from the IEEE 802.11 family of standards.

A user interface 720 may be driven by a PPH detection controller executed on a computer such as the processing system 100 communicating over the network 202. A user of the postpartum haemorrhage detection system can interact with the user interface 720 through a user input device such as a mouse 725. The user interface 720 may display device information for the PPH monitor, such as the device information including charge status of the medical device, connection and data transmission status of each sensor of the electrode assembly, service history and status, device identification, and other general information about the PPH monitor and components. The user interface 720 may also display clinical information including a diagnostic outcome of postpartum haemorrhage analysis, maternal heart rate, current heart rate, contraction information and current contraction information. Historical values of the measure may also be displayed. The charge station 710 and the user interface 720 sit on a cart 740 which may include an assembly area 730 where a PPH monitor comprising an electrode assembly and a medical device may be assembled for use on a patient.

The PPH detection controller driving the user interface 720 may receive and store raw data transmitted from the medical device, and analyse the data using machine learning and signal processing techniques. Results from the analysis may be displayed on the user interface 720.

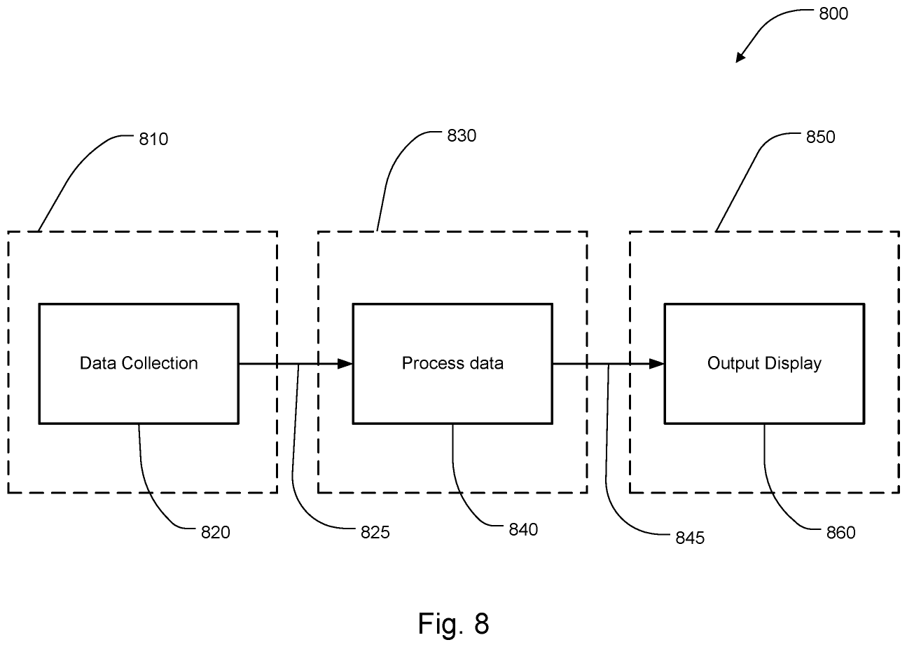

A data processing overview for data from the PPH monitor will now be described in relation to FIG. 8 which shows a PPH data processing process 800 for a patient. The PPH data processing process 800 has three parts. The first part is executed on a PPH monitor 810 where a data collection process 820 occurs. The data collection process 820 generates patient data from sensors of the PPH monitor 810. The sensors may be one or more sensors of an electrical potential sensor, a movement sensor, a deformation sensor or a patient response sensor. The PPH monitor 810 has a communication module that transmits patient data 825 to a PPH detection controller 830 using a wired or wireless connection.

The PPH detection controller 830 receives the patient data 825 as input and analyses the patient data 825 in a process data process 840. In one example of the process data process 840, the patient data 825 is analysed by a machine learning model or a trained classifier and a likelihood value produced. The likelihood value may be compared to a PPH threshold to determine if postpartum haemorrhage is likely. If the likelihood value is less than the threshold then postpartum haemorrhage is unlikely, when the likelihood value is above the threshold then PPH is likely. In one example, the likelihood should be above the PPH threshold for at least a predetermined time before PPH is determined to be likely. The output from the PPH detection controller 830 is a PPH likelihood indication 845 that may be sent to a display 850 attached to the PPH detection controller 830. The PPH likelihood indication 845 may be displayed on the display 850 as in an output process 860. In one example, the output process 860 may provide a visible and/or audible alarm on the display 850 when PPH is likely. In one example, the output process 860 may provide a binary display indicating that PPH is likely or unlikely. For example, the output process 860 may generate a status with green for PPH unlikely and red for PPH likely. In another example, the output process 860 may provide a likelihood value display on the display 850 such as a text or graphical display. Examples of a graphical display include a time-based graph where the likelihood value is graphed over time. Another example may be a gauge style display where a fill or needle displays the likelihood value. Colour variations may also be used in addition or instead of other displays.

While the display 850 is described above as being attached to the PPH detection controller 830, in one example, the display 850 may be another device such as a tablet, smartphone, or other computing device. In one example, the PPH detection controller 830 may be integrated into the PPH monitor 810. The PPH likelihood indication 845 may be transmitted to another device where the PPH likelihood is communicated to a clinician. The PPH monitor 810 may also be configured to provide the alarm if PPH is determined to be likely via an audible tone from an inbuilt speaker and/or through an integrated display on the PPH monitor 810. When the PPH monitor 810 has facilities to provide an alarm, both the PPH detection controller 830 and the display 850 may be integrated into the PPH monitor 810, e.g., in its housing. In one example, the PPH monitor 810 may include the PPH detection controller 830 and the display 850 with the PPH likelihood indication 845 also transmitted to one or more additional devices, such as a smartphone, tablet or other computing device.

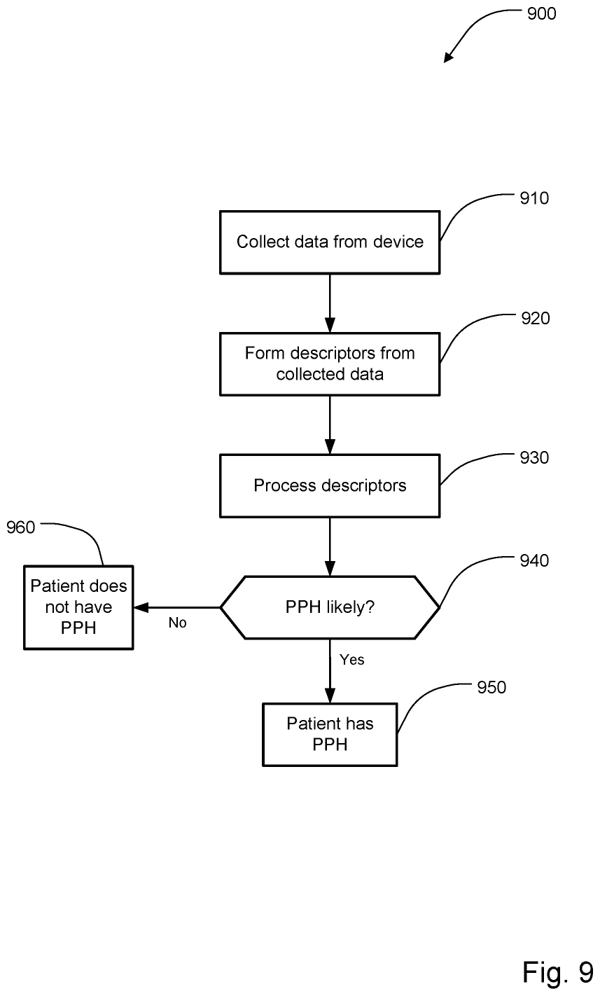

A PPH detection method 900 (also referred to as a “PPH detection process”) will now be described in relation to FIG. 9. The PPH detection method 900 may be practiced on a PPH detection controller executed by a computer such as the processing system 100 communicating over a network 202. The PPH detection method 900 takes/receives sensor input from a PPH monitor and provides an indication to a user, such as a clinician, that postpartum haemorrhage is likely or unlikely. Typically the results from the PPH detection method 900 are displayed on a monitor attached to the PPH detection controller.

At a data collection step 910 the PPH detection controller, connected to a PPH monitor using a wireless or wired interface, receives patient data from the PPH monitor sensors. The sensor may be one or more of an electrical potential sensor, a movement sensor, a deformation sensor or a patient response sensor, such as a temperature sensor.

At a descriptor generation step 920 the PPH detection controller forms high dimensional descriptors from the patient data for use in a machine learning model. The patient data may be processed in one or more steps to form the descriptors. Examples of how the descriptors are formed will be described below.

Next, as a descriptor processing step 930, the descriptors from the descriptor generation step 920 are processed by the machine learning model that has been trained using previous patient data. The output of the descriptor processing step 930 may be an estimated likelihood of PPH or a classification based on likelihood determined by the machine learning model. Examples of the machine learning models include support vector machines, neural networks, decision trees or k-Nearest Neighbours. Alternatively, both descriptor generation step 920 and descriptor processing step 930 can be performed using the same machine learning model. That is, the descriptor generation step 920 and descriptor processing step 930 may be combined and the patient data passed into a model where an initial part of the model generates descriptors and a latter part of the model processes the descriptors to produce an output of a likelihood or a classification based on likelihood.

At a PPH likely decision step 940 the output of the descriptor processing step 930 is used to decide if the patient is likely to have postpartum haemorrhage or is unlikely. When the output of the descriptor processing step 930 is a likelihood, then the likelihood may be compared to a predetermined threshold. If the likelihood is equal to or greater than the predetermined threshold then the PPH detection method 900 proceeds to a PPH likely output 950 where an indication that postpartum haemorrhage is likely is displayed by the PPH detection controller to a user. If the likelihood is less than the predetermined threshold then the PPH detection method 900 proceeds to a PPH unlikely display 960 where the PPH detection controller displays an indication to a user that postpartum haemorrhage is unlikely.

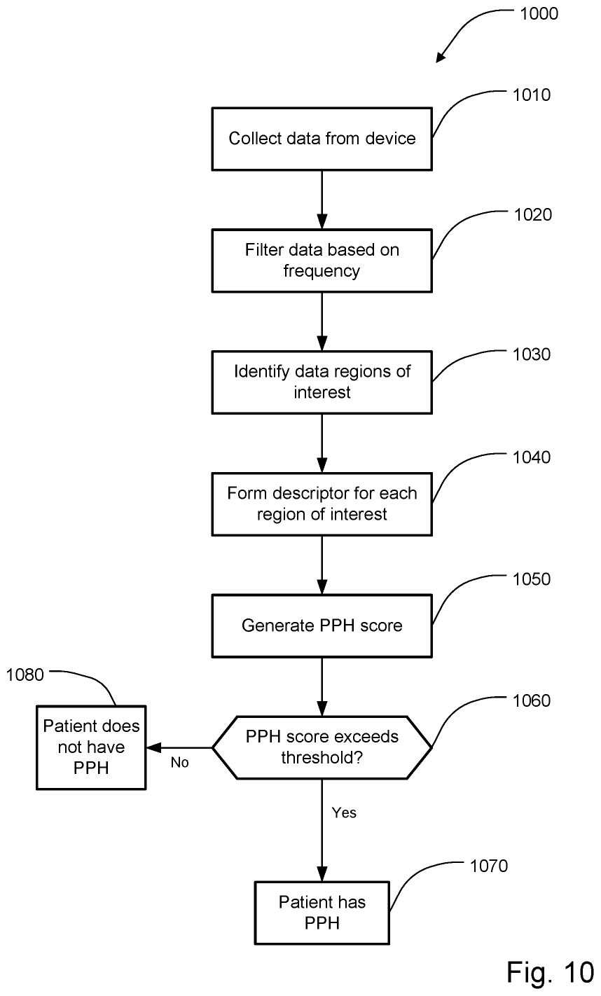

A PPH detection method 1000 (also referred to as a “PPH detection process”) will now be described in relation to FIG. 10. The PPH detection method 1000 may be practiced on the PPH detection controller executed by a computer such as the processing system 100 communicating over a network 202. The PPH detection method 1000 takes/receives, as input the data collected from the PPH monitor, and outputs a likelihood of a postpartum haemorrhage occurring to the patient. Once the PPH detection method 1000 executes, the method may detect a likelihood of PPH and notify a user. The user may then take steps to treat the PPH.

The PPH detection method 1000 starts with a data collection step 1010 where data is transmitted from the PPH monitor to the PPH detection controller. The PPH detection controller processes the data from the medical device at a data filter step 1020. The data filter step 1020 takes the data and performs a number of different processing steps. In one example, the processing may be the same for different data types received from each of the PPH monitor sensors. Alternatively, the processing may vary based on a sensor type that collected the data. For example, frequency analysis may be used on the data from each of the sensors and a band pass filter applied to select the data according to a frequency band. For electrical potential data, such as data from an EMG sensor, the data may be extracted with a frequency range between 1 Hz to 20 Hz, 1 Hz to 10 Hz or 0.05 Hz to 50 Hz. Movement sensor, such as an accelerometer, may be processed in a similar manner with the frequencies ranges from 0.5 Hz to 1 Hz, 0.1 Hz to 2 Hz or 0.05 Hz to 1 Hz. Patient response sensors, such as a temperature sensor, may also be analysed in a similar manner with a band of frequencies selected such as 0.5 Hz to 1 Hz, 0.1 Hz to 0.75 Hz or 0.05 to 1 Hz. Alternatively, frequencies above 0.05 Hz may be selected. Deformation sensors, such as a flex sensor, may be used with a band of frequencies selected such as 0.5 Hz to 1 Hz, 0.1 Hz to 0.75 Hz or 0.05 to 1 Hz. Alternatively, frequencies above 0.05 Hz or 0.01 Hz may be selected.

Next, the PPH detection method 1000 uses the processed data at an identify regions of interest step 1030. A region of interest is part of the data received from the medical device that may contain information that allows for determination of a likely PPH. Different operations may be performed at the identify regions of interest step 1030. In one example, electrical potential data is rectified and smoothed using a window with a size such as between 5 and 20 seconds, such as 5, 10, 15 or 20 seconds. The results are summed across electrical potential channels to produce a signal for further processing, where, in one example, each electrical potential channel is information collected from an electrical potential sensor. A noise floor is calculated for the signal and clipped to always be positive. The signal is then suppressed so the amplitude of the suppressed signal is below the calculated noise floor before being normalised by the number of valid electrical potential channels. Values of the output are suppressed if they are below a set threshold before being scaled so that the final signal is between 0 and 1. A threshold is applied to select regions of interest. For example, the threshold is used to select any final signal above 0.7, 0.8 or 0.9. Regions of interest are selected based on the threshold and for regions where the final signal exceeds the threshold for a predetermined window of time, such as 3, 4, 5, 6, 7 or 8 second.

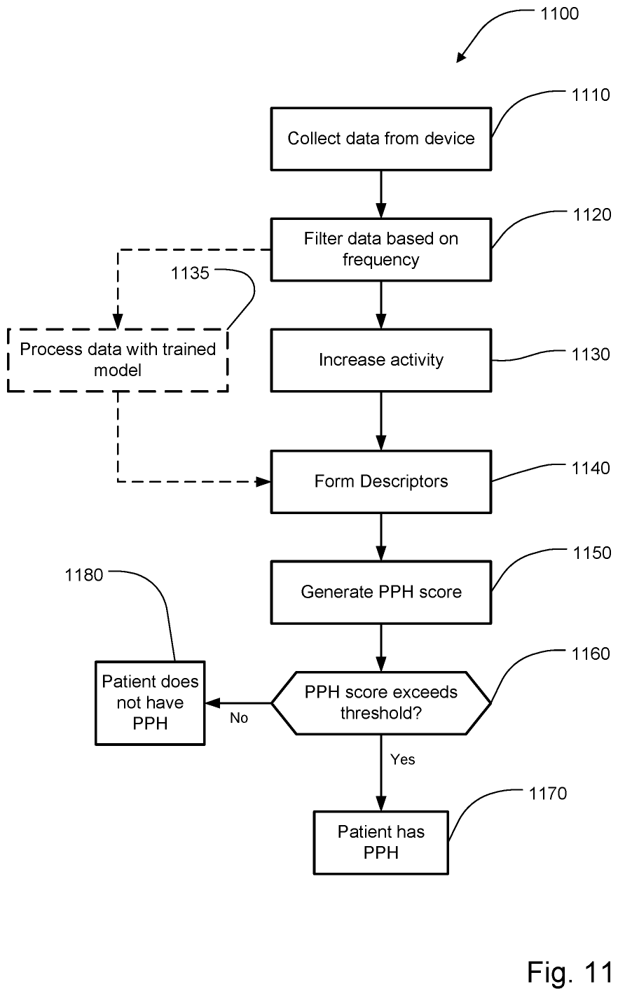

While the identify regions of interest step 1030 is described in relation to electrical potential data, similar processes may be performed for other data types such as patient response, movement and deformation sensor data. This will be described below in relation to FIG. 11.

The regions of interest from the identify regions of interest step 1030 are used at a generate descriptors step 1040. The generated descriptors are high dimensional descriptors that are used in a nonlinear model to determine likelihood of postpartum haemorrhage occurring. Generation of the high dimensional descriptors will be described by way of example for electrical potential data, such as from an EMG sensor, where the following three processes occur. The process may occur sequentially or in parallel. In a first process, the electrical potential data is normalised using a sliding window with a size between 1 and 20 seconds, such as 1, 5, 10, 15 or 20. A frequency analysis is performed on each electrical potential channel and a number of frequencies are selected. The frequencies selected are between the frequency range used in the data filter step 1020 operation, such as between 1 Hz and 20 Hz, 1 Hz to 10 Hz or 0.05 Hz to 50 Hz. A magnitude of each of the selected the frequencies is calculated across all the electrical potential channels and normalised by the number of valid electrical potential channels, where a valid electrical potential channel is a channel that is producing measurements within acceptable boundaries. The results are of size N by J, where N is the number of data measurements in a current region of interest and J is a number of frequencies selected in the analysis.

A second process performs a sliding window measure of similarity using a similarity measure such as a Euclidean distance, cosine similarity, or Kullback-Leibler divergence. The similarity measure has templates between 0.05 Hz and 5 Hz and is conducted on all electrical potential channels. The results of the similarity measure are summed across all electrical potential channels to produce an analysable result. The results are of size N by K. where N is the number of data points in the region of interest and K is the number of frequency templates used. The number of frequencies used in the first and second process may be the same or may differ, however the number of data points in the first and second process are the same.

The third process applies an energy operator, such as a Teager-Kaiser energy operator or Root Mean Square energy operator to each channel of the electrical potential data. The output of the energy operator has a size of N by L, where N is the number of data points region of interest and L is the number of electrical potential channels.

At completion of the three processes, statistical measures are calculated for each of the regions of interest and concatenated together to produce a descriptor with dimensions of A*(J+K+L) where J, K and L are described above in each of the processes and A is a number of statistical measures applied to the data. Examples of the statistical measures include mean, median, max, min, standard deviation, et cetera.

The descriptors from the generate descriptors step 1040 are used at a generate PPH score step 1050. The descriptors are passed into a trained nonlinear model to generate an output such as probabilities, pseudo-probabilities or scores where the output indicates the likelihood of a PPH.

The PPH detection method 1000 continues at a compare PPH score step 1060 where the output of the nonlinear model of the generate PPH score step 1050 may be smoothed over time using a filtering process. The output of the nonlinear model is compared against a threshold. If the output exceeds the threshold for a predetermined amount of time then PPH is determined to be likely. If the smoothed output of the nonlinear model is used, then the smoothed output may be compared to a predetermined threshold. If the threshold is not exceeded then the PPH detection method 1000 proceeds to a PPH not detected step 1080 where a user interface associated with the PPH detection method 1000 indicates that PPH is unlikely. If the threshold is exceeded then the PPH detection method 1000 proceeds to a PPH detected step 1070 where the user interface is updated to indicate that PPH is likely.

While the PPH detection method 900 selects regions of interest, one alternative may use all of the processed data without selecting regions of interest. However, in some examples, the accuracy of the PPH detection method 900 may be altered by using all the patient data.

An alternative PPH detection method (also referred to as a “PPH detection process”) will be described in relation to PPH detection method 1100 of FIG. 11. The PPH detection method 1100 is executed in a similar manner to the PPH detection method 1000 described above, however some of the operations of the PPH detection method 1100 differ. The first two operations of the PPH detection method 1100, a collect data step 1110 and a data filter step 1120, are performed as described above in relation to the collect data step 1010 and data filter step 1020.

A detect increased activity step 1130 receives the output of the data filter step 1120 and attempts to determine if uterine activity or a contraction metric has increased. An example of how the increased uterine activity or contraction metric is determined for electrical potential data is as follows. The electrical potential data from the data filter step 1120 may be rectified and smoothed using a window sized between 5 and 20 seconds. The smoothed data may be summed across electrical potential channels to produce a resultant signal. A noise floor of the resultant signal is calculated and clipped to be positive. Signal suppression is applied to decrease an amplitude of the resultant signal below the calculated noise floor. The suppressed data is then normalised by the number of valid electrical potential channels. Finally values below a predetermined threshold are suppressed and values above the predetermined threshold scale for a resultant signal between zero and one.

Alternatively, the detect increased activity step 1130 may be replaced and performed by a process data step 1135. The process data step 1135 may use machine learning methods, such as a nonlinear model, to determine a probability score indicating whether the input received from the data filter step 1120 indicates increased uterine activity or contractions.

A generate descriptors step 1140 may generate electrical potential descriptors in a similar manner to that described above in relation to the generate descriptors step 1040 of FIG. 10. For movement sensor data, such as acceleration data, a magnitude for movement data is calculated for each data sample to reduce data from X, Y and Z values to a single acceleration value. As a result, for each data point there is a single acceleration magnitude value. The acceleration magnitude is calculated based on the filtered data from the data filter step 1120. The acceleration magnitude is multiplied by the uterine activity metric before a smoothing filter is applied to reduce discontinuities data. The smooth data is divided by the proportion of time within a window of between 5 and 10 minutes where increased uterine activity is determined.

For deformation measurements, smoothed data from the data filter step 1120 is multiplied by the uterine activity metric from the detect increased activity step 1130 or the process data step 1135. A smoothing filter is then applied to reduce discontinuities in the data. The resultant data is divided by the proportion of time within a window of between 5 and 10 minutes where increased uterine activity is determined.

Patient response readings use the filtered data from the data filter step 1120 operation, which is multiplied by the uterine activity metric before a smoothing filter is applied to reduce discontinuities in the data. As with the deformation measurements and electrical potential data, the patient response readings are divided by the proportion of time within a window between 5 and 10 minutes where increased uterine activity is determined.

The descriptors are then concatenated together so that for each data sample there is a high dimensional descriptor formed by combining electrical potential data, movement data, deformation data and patient response data. Alternatively the descriptors may be formed using a combination of any two of the data types such as electrical potential data and movement data or deformation data and patient response data.

Next the PPH detection method 1100 moves to a generate PPH score step 1150 where a PPH score is determined in a similar manner to the generate PPH score step 1050 of FIG. 10. However, the nonlinear model of the generate PPH score step 1150 may be configured differently from the nonlinear model of the generate PPH score operation step 1050 as the nature of the high-level descriptors is different. The remaining steps of the PPH detection method 1100 are similar to the corresponding steps of the PPH detection method 1000. That is, a compare PPH score step 1160 is similar to the compare PPH score step 1060, a PPH not detected step 1180 is similar to the PPH not detected step 1080 and a PPH detected step 1170 is similar to the PPH detected step 1070.

As with the PPH detection method 1000, the PPH detection method 1100 generates output for display on a user interface indicating if a PPH is likely or unlikely.

In an alternative of the PPH detection method 1100 the generation of high dimensional descriptors may be performed as follows. The movement data, deformation data and the patient response data are processed as described in relation to the generate descriptors step 1140. However, the electrical potential data used to generate the descriptors is not processed as set out in the generate descriptors step 1140. Instead, the filtered electrical potential data from the data filter step 1120 is combined with the movement data, deformation data and patient response data. For each data sample there is a high dimensional descriptor forms using the data. The descriptor is then processed by a nonlinear model with the results compared against a predetermined threshold to determine if PPH is likely or unlikely, as described above.

### Variations

As described above, the PPH monitor has a one or more sensors. The sensors may be one or more of the same sensor types or a mixture of sensor types where each sensor type may be present one or more times. One sensor that may be used in the PPH monitors is an electrical potential sensor, such as electromyography (EMG), electrohepatogram (EHG) or electrocardiogram (ECG) sensors where an ECG also provides cardiac and uterine activity information. The PPH monitor may also use at least one deformation sensor to measure deformation at multiple points which may be determined using a sensor such as a stretch sensor or a flex sensor. The PPH monitor may also use movement sensors to provide data on movement and position which may be determined from a sensor such as an accelerometer or a gyroscope. Patient response sensors may be used to determine effort/patient response to events such as contractions. Such information may be determined using different sensors such as a temperature senor or a sensor to measure presence of sweat where the presence of sweat may be correlated to effort/patient response to event. While the above describes alternative sensors that may be used, different and/or additional sensors may also be used.

The PPH detection methods 900, 1000 and 1100 may be performed multiple times to determine an updated likelihood of postpartum haemorrhage over time. In one example, executions of one of the PPH detection methods is triggered based on changes in the sensor input values from the PPH monitor changes. In another alternative, executions of one of the PPH detection methods is triggered at a regular time interval, such as every 0.5 seconds or every 1 second.

In the above description a nonlinear model is used. Examples of nonlinear models that may be used include support vector machines, Gaussian processes, decision trees, graphical models, and neural networks such as recurrent neural networks, multilayer perceptrons, and convolutional neural networks. The nonlinear models may be trained using known techniques.

While the above PPH detection methods may include a plurality of ways of forming high dimensional descriptors for processing by a nonlinear model, the descriptors may be formed using other combinations of sensor data. In one example deformation data, patient response data and movement data may be used to form the high dimensional descriptors. Alternatively electrical potential data and one of the patient response data, deformation data and movement data may be used. Other combinations of sensor data may also be used.

### Results

Leave-One-Out Cross Validation (LOOCV) was used to assess the performance of the postpartum haemorrhage detection method. Under the LOOCV approach, data belonging to one patient was excluded for validation purposes while the remaining data for all other patients was used as part of the training set to generate the nonlinear model. The postpartum haemorrhage detection method, using the generated nonlinear model, was evaluated using the excluded subject. The process was repeated for each patient and an indication of performance obtained.

A pilot study was conducted for 44 subjects where the postpartum haemorrhage detection method and system achieved a high level of sensitivity (80%) and specificity (97%), with a positive predictive value of 92% and a negative predictive value of 90%. The postpartum haemorrhage detection method and system achieved a positive prediction value of at least 70, 80, or 90% and a negative prediction value of at least 70, 80, or 90%. A positive likelihood ratio of 23.20 is considered to be a large increase in the likelihood of having a postpartum haemorrhage after the diagnostic test has returned positive. A negative likelihood ratio of 0.21, if reported, is deemed as a moderate to large decrease in the likelihood of having a postpartum haemorrhage. The postpartum haemorrhage detection method determined 13 of the patients were likely to have PPH.

Truth

| | | PPH | No PPH |
|---|---|---|---|
| Postpartum | PPH | 12 | 1 |
| haemorrhage | No PPH | 3 | 28 |
| detection method | | | |

Of the 3 subjects who experienced an undetected postpartum haemorrhage, all occurred after a forceps delivery with episiotomy and documented genital tract trauma. Given that the cause of these cases of PPH was due to trauma resulting from surgical intervention, it would not be expected to detect any indications of postpartum haemorrhage prior to the event. Approximately 20% of PPH cases are due to trauma so the occurrence of three surgical cases of PPH in the 15 postpartum haemorrhage cases is consistent.

Advantages and Interpretation

The described postpartum haemorrhage detection method provides a means for determining a likelihood of postpartum haemorrhage. The PPH monitor is simple to use and may provide clinicians with effective predictions of the likelihood of PPH occurring. With such an indication, clinicians may be able to take suitable steps for effective treatment of PPH.

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). Thus, a set includes at least one element. In general, an element of a set can include or be one or more portions of a system, an apparatus, a device, a structure, an object, a process, a procedure, physical parameter, or a value depending upon the type of set under consideration.

The figures included herewith show aspects of non-limiting representative embodiments in accordance with the present disclosure, and particular structural elements shown in the figures may not be shown to scale or precisely to scale relative to each other. The depiction of a given element or consideration or use of a particular element number in a particular figure or a reference thereto in corresponding descriptive material can encompass the same, an equivalent, an analogous, categorically analogous, or similar element or element number identified in another figure or descriptive material associated therewith. The presence of “/” in a figure or text herein is understood to mean “and/or” unless otherwise indicated, i.e., “A/B” is understood to mean “A” or “B” or “A and B”. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The term “essentially all” or “substantially” can indicate a percentage greater than or equal to 50%, 60%, 70%, 80%, or 90%, for instance, 92.5%, 95%, 97.5%, 99%, or 100%.

Many modifications will be apparent to those skilled in the art without departing from the scope of the present invention.

The reference in this specification to any prior publication (or information derived from the prior publication), or to any matter which is known, is not, and should not be taken as an acknowledgment or admission or any form of suggestion that the prior publication (or information derived from the prior publication) or known matter forms part of the common general knowledge in the field of endeavour to which this specification relates.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

1. A monitoring system configured for determining risk of postpartum haemorrhage to a patient, the monitoring system comprising:
an electrical potential sensor for collecting patient data;
at least one electrode for attaching the electrical potential sensor to a body of the patient; and
a communications module for transmitting the patient data to a detection controller,
wherein the detection controller is configured to determine the risk of the postpartum haemorrhage based on the patient data.

2. The monitoring system according to claim 1, wherein the electrical potential sensor is a sensor selected from the set of sensors consisting of an electromyography (EMG) sensor, an electrohepatogram (EHG) sensor and electrocardiogram (ECG) sensor.

3. The monitoring system according to claim 1, further comprises a movement sensor.

4. The monitoring system according to claim 3, wherein the movement sensor is a sensor selected from the set of sensors consisting of an accelerometer and a gyroscope.

5. The monitoring system according to claim 1, further comprises a deformation sensor.

6. The monitoring system according to claim 5, wherein the deformation sensor is a sensor selected from the set of sensors consisting of a flex sensor and a stretch sensor.

7. The monitoring system according to claim 1, further comprising a patient response sensor.

8. The monitoring system according to claim 7, wherein the patient response sensor is a sensor selected from the set of sensors consisting of a temperature sensor and a sweat sensor.

9. The monitoring system according to claim 1, wherein the detection controller is configured to apply a machine learning model to the patient data to determine the risk of postpartum haemorrhage.

10. The monitoring system according to claim 9, wherein the machine learning model is a nonlinear model.

11. The monitoring system according to claim 9, wherein the detection controller is configured to apply the machine learning model to the patient data to determine the risk of the postpartum haemorrhage by estimating a likelihood of the postpartum haemorrhage.

12. The monitoring system according to claim 11, wherein the estimated likelihood is compared to a predetermined threshold to determine if postpartum haemorrhage is likely.

13. The monitoring system according to claim 1, wherein the detection controller includes a trained classifier configured to determine the risk of the postpartum haemorrhage.

14. The monitoring system according to claim 1, wherein the electrical potential sensor, the at least one electrode, the communications module and the detection controller are located within a housing.

15. The monitoring system according to claim 1, wherein the detection controller is located separately from the electrical potential sensor.

16. The monitoring system according to claim 1, wherein the monitoring system has a positive predictive value of at least 70%

17. The monitoring system according to claim 1, wherein the monitoring system has a negative predictive value of at least 70%.

18. The monitoring system according to claim 1, wherein the detection controller is configured to automatically perform a method comprising:
receiving the patient data from the monitoring device;
estimating a likelihood of the postpartum haemorrhage by processing the patient data from to form a plurality of descriptors for data points in the patient data, the plurality of descriptors being processed by a machine learning model to estimate the likelihood;
determining a postpartum haemorrhage risk for the patient by comparing the estimated likelihood to a predetermined threshold; and
displaying the determined postpartum haemorrhage risk to an operator.

19. A method of detecting a high risk of postpartum haemorrhage in a patient, the method comprising:
collecting patient data from a monitor having a plurality of medical electrode members attached to a body of a patient, the patient data being collected from at least one sensor type;
estimating a likelihood of the postpartum haemorrhage by processing the patient data from the at least one sensor type to form a plurality of descriptors for data points in the patient data, the plurality of descriptors being processed by a machine learning model to estimate the likelihood; and
displaying the determined postpartum haemorrhage risk to an operator.

20. The method according to claim 19, further comprising:
Determining a postpartum haemorrhage risk for the patient by comparing the estimated likelihood to a predetermined threshold.
